(19) United States
(12) Patent Application Publication
(10) Pub. No.: US 2025/0000822 A1
(43) Pub. Date: Jan. 2, 2025

(54) USE OF 2-HYDROXYBENZYLAMINE FOR THE TREATMENT OF SYSTEMIC LUPUS

(71) Applicant: Vanderbilt University, Nashville, TN (US)

(72) Inventors: David G. Harrison, Nashville, TN (US); David M. Patrick, Nashville, TN (US)

(21) Appl. No.: 18/882,674

(22) Filed: Sep. 11, 2024

Related U.S. Application Data
(63) Continuation of application No. 17/031,642, filed on Sep. 24, 2020.
(60) Provisional application No. 62/905,134, filed on Sep. 24, 2019.

(51) Int. Cl. A61K 31/137

(52) U.S. Cl. CPC A61K 31/137

(57) ABSTRACT

The invention relates to compounds, compositions, and methods for the treatment of lupus. The compounds of the present invention are isoLG savaging compounds.

### PRIOR APPLICATIONS

This application claims benefit to U.S. patent application Ser. No. 17/031,642 filed Sep. 24, 2020, which claims benefit to U.S. Patent Application No. 62/905,134, filed Sep. 24, 2019; the contents of which are incorporated herein by reference.

### GOVERNMENT SUPPORT

This invention was made with government support under grant numbers HL129941, and HL140016 awarded by the National Institutes of Health. The government has certain rights in the invention.

### BACKGROUND OF THE INVENTION

The present inventors discovered a new treatment for lupus, including systemic lupus erythematosus (SLE) using compounds of the present invention. In one embodiment, the compound is 2-hydroxybenzylamine (2-HOBA, salicylamine). In another embodiment, the compound is methyl-2-hydroxybenzylamine. In another embodiment, the compound is ethyl-2-hydroxybenzylamine.

Oxidation products arachidonic and other fatty acids, termed isolevuglandins (IsoLG) lead to formation of protein adducts that are immunogenic. The present inventors discovered that isoLG-adducted peptides are markedly enriched in monocytes from patients with SLE compared to matched healthy controls.

Systemic lupus erythematosus (SLE) is a chronic autoimmune disease that affects 300,000-1.5 million people in the U.S. (90% of whom are women) and has serious health implications. Because SLE affects multiple organ systems, patients with SLE are at elevated risk for vascular dysfunction, cardiovascular disease, renal disease, and other morbidities. Approximately 10-15% of patients will die prematurely due to complications of SLE despite treatment with currently available therapeutic options. SLE is associated with multiple immunologic abnormalities, and both innate and adaptive immune cells have been implicated in the pathogenesis of SLE. Of note, antigen-presenting cells, including dendritic cells and monocytes, are thought to present self-antigens to self-reactive T-cells that contribute to tissue damage and inflammation. T-cells that are activated by autoantigens also participate in the activation of auto-reactive B-cells. In SLE, mature B cells termed plasma cells produce pathogenic antibodies that lead to immune complex formation; these complexes deposit in nearly every organ, leading to inflammation and organ damage.

Reactive oxygen species (ROS) play critical signaling roles in cells and tissues, but excessive ROS production can lead to cell damage and inflammation and contribute to SLE pathogenesis. Patients with SLE exhibit augmented oxidation of LDL particles which may contribute to augmented cardiovascular risk. Numerous studies have reported augmented lipid peroxidation in SLE a result of ROS. Importantly, oxidized phospholipids can bind to proteins and augment their immunogenicity. In SLE, these have been shown to correlate with disease activity, suggesting a role of these modifications in the activation of the adaptive immune system. Highly reactive isolevuglandins (isoLGs) are byproducts of arachidonic acid peroxidation by ROS, and a minority of these are formed as intermediates of the cyclooxygenase pathway. IsoLGs are highly labile and form covalent bonds to lysine residues of nearby proteins, causing loss of protein function, mitochondrial and endoplasmic reticulum stress, and inflammation.

The inventers have discovered that isoLG-modified proteins are elevated in patients with SLE, and have shown that these can activate the adaptive immune system when presented by dendritic cells. Moreover, our data indicate that patients with SLE exhibit augmented accumulation of isoLG-adducts within antigen presenting cells. FIG. 1 illustrates the role of isoLGs in SLE pathogenesis.

### SUMMARY OF THE INVENTION

Disclosed is a method for treating lupus that comprises identifying a subject in need of treatment for lupus, and administering to said subject an effective isoLG scavenging amount of at least one compound of the following formula:

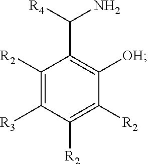

wherein R₂ is independently chosen from H, substituted or unsubstituted alkyl; R₃ is H, halogen, alkyl, alkoxy, hydroxyl, nitro; R₄ is H, substituted or unsubstituted alkyl, carboxyl; and pharmaceutically acceptable salts thereof.

In one embodiment, R₂ is independently chosen from H, ethyl, methyl. In another embodiment, the compound is 2-hydroyxbenzylamine, methyl-2-hydroyxbenzylamine, ethyl-2-hydroyxbenzylamine. In another embodiment, the compound is:

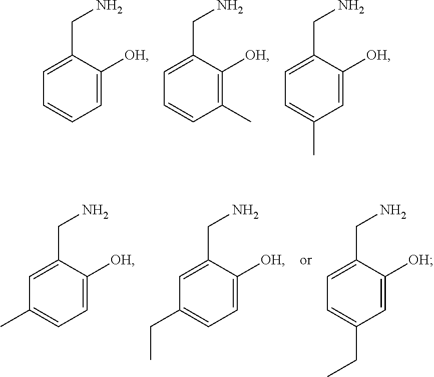

or a pharmaceutically acceptable salt thereof.

In another embodiment, the compound is:

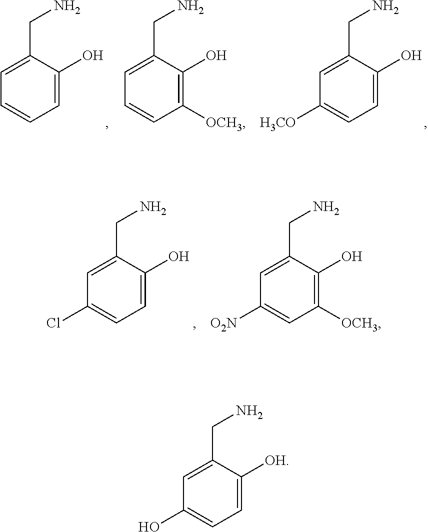

or a pharmaceutically acceptable salt thereof.

In one embodiment, the lupus is systemic lupus erythematosus.

Also, in one embodiment, the disclosed the treating step inhibits the progression of lupus. Also, in one embodiment, the disclosed treating step attenuates the severity of lupus. Also, in one embodiment, the disclosed the treating step mitigates the damaging effects of lupus in the subject. The damaging effects include abnormal sodium excretion, augmented albuminuria, and augmented glomerular injury.

In another embodiment, the compound or pharmaceutically acceptable salt thereof is administered in a composition that comprises said compound or a pharmaceutically acceptable salt thereof and a pharmaceutically acceptable carrier.

In other embodiment, the compound or pharmaceutically acceptable salt thereof is administered is co-administered with another active agent that had a known side effect of treating lupus.

Additional advantages and embodiments of the present invention will be set forth in the description which follows, and in part will be obvious from the description, or can be learned by practice of the invention. The advantages and embodiments of the present invention will be realized and attained by means of the elements and combinations particularly pointed in the appended claims. It is to be understood that both the foregoing general description and the following more detailed description are exemplary and exemplary only and are not restrictive of the invention, as claimed.

### BRIEF DESCRIPTION OF THE FIGURES

FIG. 1 shows a model of isoLG adduct induction of SLE: In antigen presenting cells, reactive oxygen species result in the formation of isoLG (yellow star) and adduction of proteins. Adducted proteins are subsequently processed into peptides, loaded in the major histocompatibility complex and presented to a T-cell. The T-cell is co-stimulated by the antigen presenting cell and proliferates.

FIG. 2A-G shows that IsoLG adducts are enriched in monocytes from patients with SLE. (A) Representative fluorescence-activated cell sorting (FACS) plots of isoLG-adduct staining in CD11c⁺ PBMCs from controls and SLE patients. Representative histograms of isoLG-adducts distribution in (B) CD11c⁺ and (C) CD11c⁺/CD86⁺ cells. Quantitation of IsoLG-adduct positive cells as a percentage of (D) CD11c⁺, (E) CD11c⁺/CD86⁺, (F) CD83⁺, and (G) CD14⁺ cells. (n=10-11, *P<0.05).

FIG. 3 is a chemical scheme demonstrating how 2-HOBA selectively scavenges isoLGs to inhibit adduct formation.

FIG. 4A-D shows that IsoLG adducts are enriched in monocytes, DCs, and plasma cells in SLE prone mice. Representative histograms revealing isoLG adduct enrichment and efficient scavenging by 2-HOBA in (A) splenic DCs and (B) circulating monocytes. Quantitation of isoLG-adduct containing cells in (C) splenic DCs (D) circulating monocytes. (n=5-9, *P<0.05, **P<0.01, ***P<0.001, ****P<0.0001).

FIG. 5A-C shows scavenging of isoLG attenuates renal inflammation in a mouse model of SLE. Single cell suspensions were prepared from freshly isolated mouse kidney via enzymatic digestion and mechanical dissociation. Live cell singlets were analyzed. Representative FACS plots are presented for (A) CD3⁺ T-cells. Quantitation of (B) CD45⁺ leukocytes and (C) CD3⁺ T-cells are represented. (n=6-8, *P<0.05, **P<0.01).

FIG. 6A-D shows that 2-HOBA attenuates renal injury and dysfunction in a mouse model of SLE. Kidneys were sectioned and stained with Jackson's silver stain; (A) representative glomeruli are presented. (B) Mice received IP injection of 4% normal saline at 10% of body weight and urine output was measured over 4 hours. (C) Urine albumin/creatine ratio. (D) Urinary NGAL. (n=6-8, *P<0.05, **P<0.01, ****P<0.0001).

FIG. 7A-D shows that 2-HOBA reduces plasma cell expansion in bone marrow and reduces IgG and anti-dsDNA antibody titers in a mouse model of SLE. (A) Representative FACS plots displaying CD138⁺ and intracellular Igκ-light-chain⁺ plasma cells from bone marrow. Quantitation of plasma cells as a percentage of CD45 cells are represented for (B) bone marrow. (C) Anti-dsDNA IgG antibody and (D) total plasma IgG at 1:100K dilution were analyzed by ELISA. (n=5-7, *P<0.05, **P<0.01, ***P<0.001, ****P<0.0001).

### DESCRIPTION OF THE INVENTION

The present inventors have discovered that compounds of the present invention are effective in the treatment of treatment of lupus, including systemic lupus erythematosus (SLE).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “subject” refers to a target of administration. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. As can be seen herein, there is overlap in the definition of treating and preventing.

As used herein, the term “diagnosed” means having been subjected to a physical examination by a person of skill, for example, a physician, and found to have a condition that can be diagnosed or treated by the compounds, compositions, or methods disclosed herein. As used herein, the phrase “identified to be in need of treatment for a disorder,” or the like, refers to selection of a subject based upon need for treatment of the disorder. For example, a subject can be identified as having a need for treatment of a disorder (e.g., a disorder related to inflammation) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “scavenger” or “scavenging” refers to a chemical substance that can be administered in order to remove or inactivate impurities or unwanted reaction products. For example, without being bound by theory or mechanism, the isoLGs irreversibly adduct specifically to lysine residues on proteins. The isoLGs scavengers of the present invention react with isoLGs before they adduct to the lysine residues. Accordingly, the compounds of the present invention “scavenge” isoLGs, thereby preventing them from adducting to proteins.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by a formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹—OA² or —OA¹—(OA²)ₐ—OA³, where “a” is an integer of from 1 to 200 and A¹, A², and A³ are alkyl and/or cycloalkyl groups.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “hydroxyl” as used herein is represented by a formula —OH.

The term “nitro” as used herein is represented by a formula —NO₂.

The term “pharmaceutically acceptable” describes a material that is not biologically or otherwise undesirable, i.e., without causing an unacceptable level of undesirable biological effects or interacting in a deleterious manner.

Examples of compounds of the present invention include, but are not limited to, compounds selected from the formula:

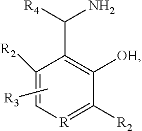

wherein: R is N or C—R₂; R₂ is independently H, substituted or unsubstituted alkyl; R₃ is H, halogen, alkyl, alkoxy, hydroxyl, nitro; R₄ is H, substituted or unsubstituted alkyl, carboxyl; and pharmaceutically acceptable salts thereof.

Further example include compounds of the following formula:

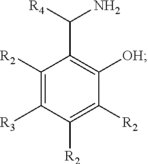

wherein: R₂ is independently chosen from H, substituted or unsubstituted alkyl; R₃ is H, halogen, alkyl, alkoxy, hydroxyl, nitro; R₄ is H, substituted or unsubstituted alkyl, carboxyl; and pharmaceutically acceptable salts thereof. In other embodiments, R₂ is independently chosen from H, ethyl, methyl.

In other embodiments, the compound may be chosen from:

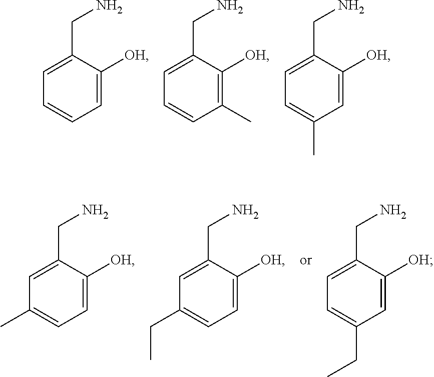

or a pharmaceutically acceptable salt thereof.

The compound may also be chosen from:

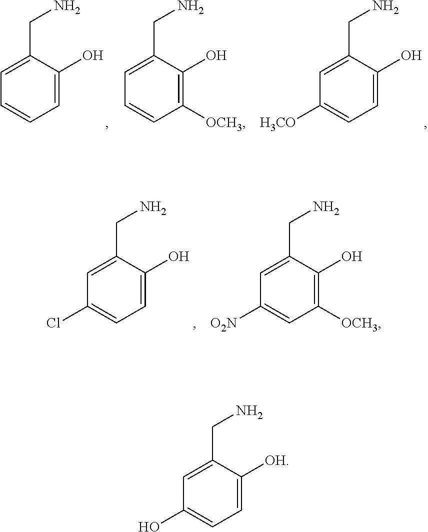

or a pharmaceutically acceptable salt thereof.

The compounds may also be chosen from:

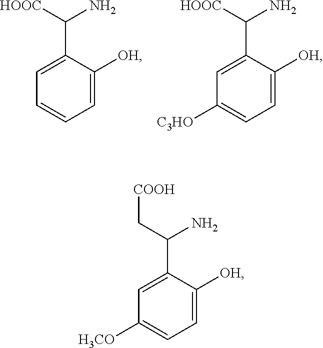

or a pharmaceutically acceptable salt thereof.

The compounds may also be chosen from

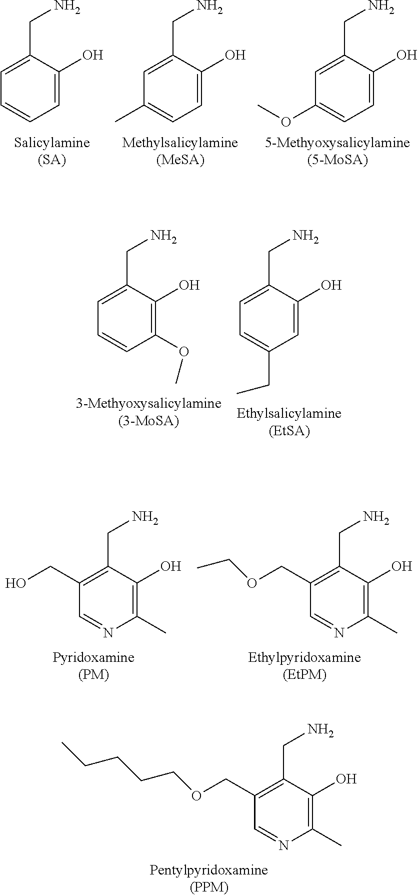

or a pharmaceutically acceptable salt thereof.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

Accordingly, one embodiment of the present invention is a method for treating lupus, comprising administering to a patient in need thereof an effective amount of at least one isoLG scavenger compound of the present invention, or a pharmaceutically acceptable salt thereof. Preferably, the compound is 2-HOBA, methyl-2-HOBA or ethyl-2-HOBA.

Another embodiment of the present invention is a method for treating lupus, including lupus erythematosus (SLE), comprising administering to a patient in need thereof an effective amount of at least one isoLG scavenger compound of the present invention, or a pharmaceutically acceptable salt thereof. Preferably, the compound is 2-HOBA, methyl-2-HOBA or ethyl-2-HOBA.

Another embodiment of the present invention is a method of mitigating the damaging effects of oxidative stress on SLE pathogenesis, comprising administering to a patient in need thereof an effective amount of at least one isoLG scavenger compound of the present invention, or a pharmaceutically acceptable salt thereof. Preferably, the compound is 2-HOBA, methyl-2-HOBA or ethyl-2-HOBA.

Another embodiment of the present invention is treatment to prevent or inhibit the progression of lupus, including lupus erythematosus (SLE), and/or including the damaging effects of lupus, comprising administering to a patient in need thereof an effective amount of at least one isoLG scavenger compound of the present invention, or a pharmaceutically acceptable salt thereof. Preferably, the compound is 2-HOBA, methyl-2-HOBA or ethyl-2-HOBA.

Another embodiment of the present invention is to attenuate and/or reverse lupus, including lupus erythematosus (SLE), comprising administering to a patient in need thereof an effective amount of at least one isoLG scavenger compound of the present invention, or a pharmaceutically acceptable salt thereof. Preferably, the compound is 2-HOBA, methyl-2-HOBA or ethyl-2-HOBA.

The administration step can include administering a compound of the present invention as part of a pharmaceutical composition. The pharmaceutical compositions of the present invention include the compound of the present invention and a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

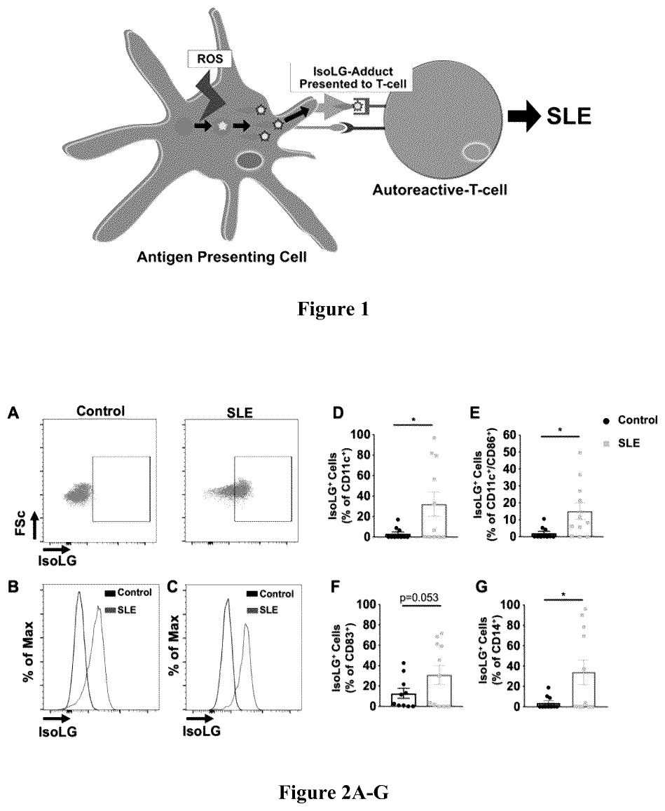

The present inventors have shown that IsoLGs are elevated in myeloid cells of SLE patients. Peripheral blood mononuclear cells (PBMCs) from SLE patients (n=11) and controls (n=10) were costained for isoLG-adducts, CD11c (dendritic cell), CD14 (monocyte), CD86 (costimulatory molecule), and CD83 (activation marker). IsoLG-adducts were enriched in CD11c⁺ and CD14⁺ monocytes from patients with SLE, suggesting isoLGs may contribute to SLE pathogenesis by participating in target cell activation by antigen presenting cells (see FIG. 2).

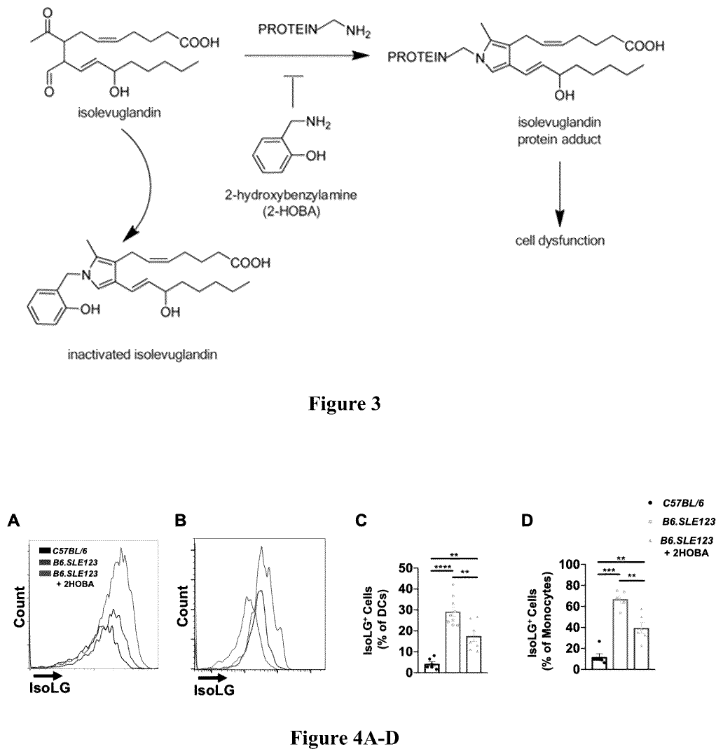

Compounds of the present invention, including 2-hydroxybenzylamine, are potent isoLG scavengers. 2-HOBA, for example, is naturally found in buckwheat seeds. 2-HOBA is highly reactive and found to react with isoLGs at a rate more than 3 orders of magnitude faster than the e-amine of lysine (see FIG. 3). By selectively reacting with isoLGs, 2-HOBA inhibits isoLG:protein adduct formation and subsequent isoLG-induced cell dysfunction. 2-HOBA dramatically protected HepG2 cells against H₂O₂-induced cytotoxicity. These data support our hypothesis that 2-HOBA effectively protect cells against the damaging effects of excess ROS.

The present inventors have also shown that 2-HOBA reduces isoLG accumulation in monocytes and dendritic cells (DCs) of SLE-prone mice. B6.SLE123 mice were treated with 2-HOBA starting at 7 weeks of age (prior to disease presentation). At 32 weeks of age, peripheral blood, spleen, and bone marrow cells were harvested from B6.SLE123 and control (C57BL/6) mice. Flow cytometry was used to detect the surface markers CD11c, IAb, CD11b, F4/80, and Ly6c and isoLG adducts. Intracellular isoLG adducts within splenic dendritic cells (see FIG. 4A) and peripheral blood monocytes (see FIG. 4B) were markedly increased in B6.SLE123 compared to control mice. However, 2-HOBA administration significantly attenuated isoLG accumulation in both monocytes and DCs.

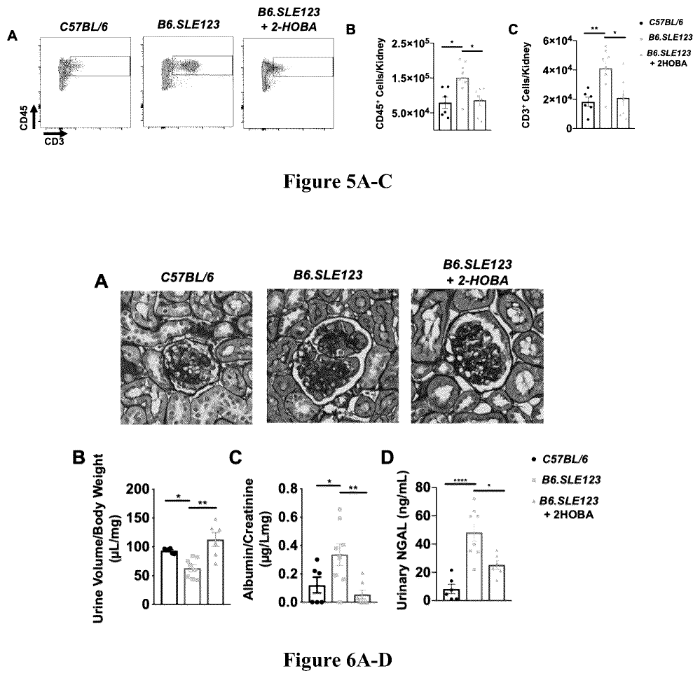

The present inventors have also shown that 2-HOBA reduces SLE-associated inflammation. Like patients with SLE, B6.SLE123 mice exhibit renal inflammation and functional impairment; these are associated with T-cell accumulation within the renal parenchyma. Renal inflammation was markedly reduced by 2-HOBA, as evidenced by a reduction in renal CD45⁺ cells and CD3⁺ T-cells in 2-HOBA-treated B6.SLE123 mice (see FIG. 5A-C). Similar results were obtained when experiments were repeated in NZBWF1/J mice.

The present inventors have also shown that 2-HOBA protects against SLE-associated renal injury and dysfunction. Patients with SLE may exhibit abnormal sodium excretion, augmented albuminuria, and augmented glomerular injury secondary to immune complex deposition. 2-HOBA markedly improved glomerular hypercellularity and immune complex deposition in SLE-prone mice (FIG. 6A). To assess renal function, 2-HOBA treated and untreated B6.SLE123 animals were injected with 4% normal saline at a volume equal to 10% of their body weight, and urine volume was measured over 4 hours. Urine volume was significantly reduced in B6.SLE123 mice compared to controls, but improved with 2-HOBA (FIG. 6B). Albumin to creatinine ratio and NGAL excretion (a marker of glomerular injury) were significantly elevated in B6.SLE123 mice, but also improved with 2-HOBA treatment (FIG. 6C-D). Similar results were obtained when experiments were repeated with NZBWF1/J mice. These data suggest that isoLGs play an important role in renal inflammation, injury, and immune complex deposition in SLE and that 2-HOBA can prevent renal injury and dysfunction.

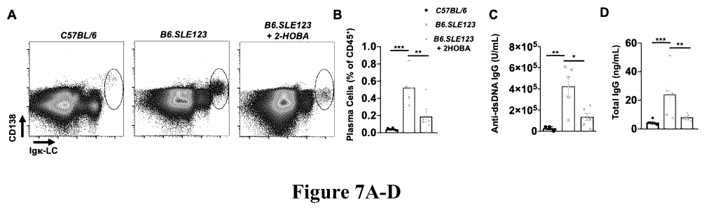

The present inventors have also shown that 2-HOBA reduces bone marrow plasma cell expansion and anti-dsDNA antibody titers. SLE is associated with an expansion of plasma cells and an amplification of auto-antibodies. Autoreactive plasma cells are believed to play an important role in SLE by generating antibodies that lead to the creation of immune complexes which deposit in peripheral tissues. Flow cytometry was performed to identify CD138⁺/Igκ-light-chain⁺ plasma cells in spleen and bone marrow cell preparations from B6.SLE123 animals treated with vehicle or 2-HOBA. B6.SLE123 animals had significant accumulation of spleen and bone marrow plasma cells which were attenuated by 2-HOBA (FIG. 7A-C). Importantly, B6.SLE123 animals treated with 2-HOBA also exhibited markedly lower plasma anti-dsDNA antibody and total IgG antibody titers (FIG. 7C-D). These data demonstrate that scavenging of isoLGs with 2-HOBA attenuates both plasma cell accumulation and autoantibody elaboration in SLE. Similar results were obtained when experiments were repeated with NZBWF1/J mice.

The data described herein, including the Figures, clearly demonstrate a role for isoLGs in SLE disease pathogenesis, including antigen presentation and subsequent B-cell activation, a hallmark of SLE. Importantly, they also provide remarkable evidence of the protective effect of 2-HOBA in SLE pathogenesis.

As stated above, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention can comprise a compound of the invention (or pharmaceutically acceptable salts thereof) as an active ingredient, a pharmaceutically acceptable carrier, and optionally one or more additional therapeutic agents or adjuvants.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

It is understood that the disclosed compositions can be prepared from the disclosed compounds. It is also understood that the disclosed compositions can be employed in the disclosed methods of using.

Further disclosed herein are pharmaceutical compositions comprising one or more of the disclosed lupus treating compounds and a pharmaceutically acceptable carrier.

Accordingly, the pharmaceutical compositions of the present invention include those that contain one or more other active ingredients, in addition to a compound of the present invention.

The above combinations include combinations of a disclosed compound not only with one other active compound, but also with two or more other active compounds. Likewise, disclosed compounds may be used in combination with other drugs that are used in the prevention, treatment, control, amelioration, or reduction of risk of the diseases or conditions for which disclosed compounds are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient can be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000 and any amount in-between, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent may be coadministered, either in concomitant therapy or in a fixed combination.

The invention is especially useful, and meets a long-felt need because existing treatments for SLE are limited by their potential for serious side effects and toxicity. Commonly used therapeutics include anti-inflammatories/pain relievers, corticosteroids, antimalarials, and immune modulators. Long-term use of anti-inflammatories can cause gastrointestinal complications and liver/kidney damage. Corticosteroids, such as prednisone, have several side effects, including weight gain, increased infection risk, induction of diabetes, bone damage, and muscle weakness. High doses of antimalarial drugs can cause retinal toxicity and alter vision. Immunosuppressive drugs are used in severe cases of SLE, or when other treatments cannot adequately control symptoms. Many of these drugs were originally indicated as chemotherapy or anti-rejection drugs, and they are associated with serious side effects. All reduce a patient's ability to fight off infections, and other serious drug-specific side effects exist, including kidney and liver damage and infertility. The newest FDA-approved drug for lupus is belimumab, which is a monoclonal antibody that disrupts B lymphocyte activation. Belimumab also carries an increased risk of infection, and is only approved for patients receiving standard therapy, compounding the risk of side effects. Beyond the serious side effects, many of the existing therapeutic options create other challenges for patients, as many are very expensive and must be regularly injected or infused rather than taken orally.

Thus, there is a long-felt need for a new SLE therapeutic specifically aimed at interrupting the disease process with fewer side effects/risks. The compound of the present invention have the potential to provide a much safer therapeutic option for patients with SLE. Rather than suppressing the immune system, compounds of the present invention selectively target dysfunction within the immune cells. As compounds of the present invention, including 2-HOBA, selectively scavenge isoLGs, it has the unique potential to mitigate the damaging effects of oxidative stress on SLE pathogenesis without interfering with normal ROS signaling. There have been in vitro safety studies of 2-HOBA (cytotoxicity, mutagenicity, CYP induction, hERG inhibition, and plasma and red blood cell binding in multiple species). Several animal toxicity studies were performed, including an acute study in rats, short term (28-day) studies in mice and rats, and subchronic (90-day) studies in rats and rabbits. The first-in-human study of 2-HOBA, for example, found that single doses of 2-HOBA up to 825 mg (the highest dose tested) were safe and well-tolerated in healthy human volunteers. The first multiple dose study of 2-HOBA, testing doses of 500 and 750 mg TID for 2 weeks, was recently completed in healthy human volunteers. Multiple doses of 2-HOBA were well tolerated; there were no serious adverse events and accumulation of 2-HOBA and its primary metabolite were low. See Pitchford L M. Driver P M, Fuller J C Jr, et al. Safety, tolerability, and pharmacokinetics of repeated oral doses of 2-hydroxybenzylamine acetate in healthy volunteers: a double-blind, randomized, placebo-controlled clinical trial. BMC Pharmacol Toxicol. 2020; 21(1):3. Published 2020 Jan. 6. doi:10.1186/s40360-020-0382-y. To date, no serious adverse effects or potential issues have been observed in vitro, in animals, or in humans.

In addition to its superior safety profile, compounds of the present invention are also desirable for its feasibility of use. While an option for administration, compounds of the present invention do not have to be injected or infused, as they are orally bioavailable. Further, compounds of the present invention have a long shelf life at room temperature (≥3 years). Compounds of the present invention also can be manufactured at a substantially lower cost compared to biologic treatments, which will further lower patient burden and ensure access.

In another embodiment of the present invention, the compounds of the present invention can be co-administered to a patient in need thereof with another active ingredient that has a known side effect of treating lupus, including systemic lupus erythematosus.

That is, a compound of the present invention can be administered alone or in combination with an effective amount of at least one additional agent which is traditionally used in the treatment of system lupus erythematosus. These agents include, for example, non-steroidal anti-inflammatory drugs (NSAIDs) including traditional NSAIDs, COX-2 inhibitors and salicylates (such as aspirin), anti-malarials such as hydroxychloraquine, quinacrine, corticosteroids such as prenisone (Deltasone), betamethasone (Celestone), methylprednisolone acetate (Medrol, Depo-Medrol), hydrocortisone Cortef, Hydrocortone) and dexamethasone (Decadron, Hexadrol), among others and immunosuppressants such as methotrexate (Rhematrex), cyclophosphamide (cytoxan), Azathioprine (Imuran) and mycophenolate mofetil (MMF, also Cellsept),

“Combined” or “in combination” or “combination” should be understood as a functional co-administration, wherein some or all compounds may be administered separately, in different formulations, different modes of administration (for example subcutaneous, intravenous or oral) and different times of administration. The individual compounds of such combinations may be administered either sequentially in separate pharmaceutical compositions as well as simultaneously in combined pharmaceutical compositions.

All publications mentioned herein, including those listed below, are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.
1 Wallace, D. J. & Hahn, B. H. Dubois' lupus erythematosus and related syndromes. 8th edn, (Elsevier Saunders, 2013).
2 Yaniv, G. et al. A volcanic explosion of autoantibodies in systemic lupus erythematosus: a diversity of 180 different antibodies found in SLE patients. Autoimmun Rev 14, 75-79, doi:10.1016/j.autrev.2014.10.003 (2015).
3 Gaipl, U. S. et al. Clearance deficiency and systemic lupus erythematosus (SLE). J Autoimmun 28, 114-121, doi:10.1016/j.jaut.2007.02.005 (2007).
4 Mackern-Oberti, J. P. et al. Role of dendritic cells in the initiation, progress and modulation of systemic autoimmune diseases. Autoimmun Rev 14, 127-139, doi:10.1016/j.autrev.2014.10.010 (2015).
5 Shah, D., Mahajan, N., Sah, S., Nath, S. K. & Paudyal, B. Oxidative stress and its biomarkers in systemic lupus erythematosus. J Biomed Sci 21, 23, doi:10.1186/1423-0127-21-23 (2014).
6 Park, J. K. et al. Altered lipoproteins in patients with systemic lupus erythematosus are associated with augmented oxidative stress: a potential role in atherosclerosis. Arthritis Res Ther 18, 306, doi:10.1186/s13075-016-1204-x (2016).
7 Lightfoot, Y. L., Blanco, L. P. & Kaplan, M. J. Metabolic abnormalities and oxidative stress in lupus. Curr Opin Rheumatol 29, 442-449, doi:10.1097/BOR.0000000000000413 (2017).
8 Khan, F. et al. Immunochemical studies on HNE-modified HSA: Anti-HNE-HSA antibodies as a probe for HNE damaged albumin in SLE. Int J Biol Macromol 86, 145-154, doi:10.1016/j.ijbiomac.2016.01.053 (2016).
9 Wang, G. et al. Significance of Lipid-Derived Reactive Aldehyde-Specific Immune Complexes in Systemic Lupus Erythematosus. PLoS One 11, e0164739, doi:10.1371/journal.pone.0164739 (2016).
10 Brame, C. J., Salomon, R. G., Morrow, J. D. & Roberts, L. J. Identification of extremely reactive gamma-ketoaldehydes (isolevuglandins) as products of the isoprostane pathway and characterization of their lysyl protein adducts. J. Biol. Chem 274, 13139-13146 (1999).
11 Morrow, J. D. et al. A series of prostaglandin F2-like compounds are produced in vivo in humans by a non-cyclooxygenase, free radical-catalyzed mechanism. Proc. Natl. Acad. Sci. U.S.A 87, 9383-9387 (1990).
12 Salomon, R. G. & Miller, D. B. Levuglandins: isolation, characterization, and total synthesis of new secoprostanoid products from prostaglandin endoperoxides. Adv. Prostaglandin Thromboxane Leukot. Res 15, 323-326 (1985).
13 Davies, S. S. et al. Effects of reactive gamma-ketoaldehydes formed by the isoprostane pathway (isoketals) and cyclooxygenase pathway (levuglandins) on proteasome function. FASEB J 16, 715-717 (2002).
14 Miyashita, H. et al. Lysine pyrrolation is a naturally-occurring covalent modification involved in the production of DNA mimic proteins. Sci Rep 4, 5343, doi:10.1038/srep05343 (2014).
15 Kirabo, A. et al. DC isoketal-modified proteins activate T cells and promote hypertension. J Clin Invest 124, 4642-4656, doi:10.1172/JCI74084 (2014).
16 Lob, H. E., Schultz, D., Marvar, P. J., Davisson, R. L. & Harrison, D. G. Role of the NADPH oxidases in the subfornical organ in angiotensin II-induced hypertension. Hypertension 61, 382-387, doi:10.1161/HYPERTENSIONAHA.111.00546 (2013).
17 Montezano, A. C. & Touyz, R. M. Reactive oxygen species, vascular Noxs, and hypertension: focus on translational and clinical research. Antioxid Redox Signal 20, 164-182, doi:10.1089/ars.2013.5302 (2014).
18 Amarnath, V., Amarnath, K., Amarnath, K., Davies, S. & Roberts, L. J. Pyridoxamine: an extremely potent scavenger of 1,4-dicarbonyls. Chem Res. Toxicol 17, 410-415 (2004).
19 Davies, S. S. et al. Pyridoxamine analogues scavenge lipid-derived gamma-ketoaldehydes and protect against H2O2-mediated cytotoxicity. Biochemistry 45, 15756-15767 (2006).
20 Trott, D. W. et al. Oligoclonal CD8+ T cells play a critical role in the development of hypertension. Hypertension 64, 1108-1115, doi:10.1161/HYPERTENSIONAHA.114.04147 (2014).
21 Saleh, M. A. et al. Lymphocyte adaptor protein LNK deficiency exacerbates hypertension and end-organ inflammation. J Clin Invest 125, 1189-1202, doi:10.1172/JCI76327 (2015).
22 Wu, J. et al. Immune activation caused by vascular oxidation promotes fibrosis and hypertension. J Clin Invest 126, 1607, doi:10.1172/JCI87425 (2016).
23 Taylor, E. B., Barati, M. T., Powell, D. W., Turbeville, H. R. & Ryan, M. J. Plasma Cell Depletion Attenuates Hypertension in an Experimental Model of Autoimmune Disease. Hypertension 71, 719-728, doi:10.1161/HYPERTENSIONAHA.117.10473 (2018).
24 Fuller, J. C., Jr. et al. In vitro safety pharmacology evaluation of 2-hydroxybenzylamine acetate. Food Chem Toxicol 121, 541-548, doi:10.1016/j.fct.2018.09.047 (2018).
25 Pitchford, L. M., Smith, J. D., Abumrad, N. N., Rathmacher, J. A. & Fuller, J. C., Jr. Acute and 28-day repeated dose toxicity evaluations of 2-hydroxybenzylamine acetate in mice and rats. Regul Toxicol Pharmacol, doi:10.1016/j.yrtph.2018.07.026 (2018).
26 Fuller, J. C., Jr., Pitchford, L. M., Abumrad, N. N. & Rathmacher, J. A. Subchronic (90-day) repeated dose toxicity study of 2-hydroxybenzylamine acetate in rats. Regul Toxicol Pharmacol 99, 225-232, doi:10.1016/j.yrtph.2018.09.025 (2018).
27 Fuller, J. C., Jr., Pitchford, L. M., Abumrad, N. N. & Rathmacher, J. A. Subchronic (90-day) repeated dose oral toxicity study of 2-hydroxybenzylamine acetate in rabbit. Regul Toxicol Pharmacol 100, 52-58, doi:10.1016/j.yrtph.2018.10.017 (2018).
28 Pitchford, L. M. et al. First-in-human study assessing safety, tolerability, and pharmacokinetics of 2-hydroxybenzylamine acetate, a selective dicarbonyl electrophile scavenger, in healthy volunteers. BMC Pharmacol Toxicol 20, 1, doi:10.1186/s40360-018-0281-7 (2019).
29 Otaki, N. et al. Identification of a lipid peroxidation product as the source of oxidation-specific epitopes recognized by anti-DNA autoantibodies. J Biol Chem 285, 33834-33842, doi:10.1074/jbc.M110.165175 (2010).
30 Zagol-Ikapitte, I. A. et al. Determination of the Pharmacokinetics and Oral Bioavailability of Salicylamine, a Potent gamma-Ketoaldehyde Scavenger, by LC/MS/MS. Pharmaceutics 2, 18-29 (2010).
31 Food and Drug Administration. in Center for Drug Evaluation and Research (U.S. Food and Drug Administration, 2005).

The invention thus being described, it would be obvious that the same can be varied in many ways. Such variations that would be obvious to one of ordinary skill in the art is to be considered as being bard of this disclosure.

Unless otherwise indicated, all numbers expressing quantities of ingredients, properties such as reaction conditions, and so forth used in the Specification are to be understood as being modified in all instances by the term “about.” Accordingly, unless indicated by the contrary, the numerical parameters set forth in the Specification and Claims are approximations that may vary depending upon the desired properties sought to be determined by the present invention.

Notwithstanding that the numerical ranges and parameters setting forth the broad scope of the invention are approximations, the numerical values set forth in the experimental sections or the example sections are reported as precisely as possible. Any numerical value, however, inherently contain certain errors necessarily resulting from the standard deviation found in their respective testing measurements.

We claim:

1. A method for treating lupus, comprising:
identifying a subject in need of treatment for lupus;
administering to said subject an effective isoLG scavenging amount of at least one compound of the following formula: 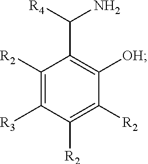
wherein:
R₂ is independently chosen from H, substituted or unsubstituted alkyl;
R₃ is H, halogen, alkyl, alkoxy, hydroxyl, nitro;
R₄ is H, substituted or unsubstituted alkyl, carboxyl; and pharmaceutically acceptable salts thereof.

2. The method of claim 1, wherein R₂ is independently chosen from H, ethyl, methyl.

3. The method of claim 1, wherein the compound is 2-hydroyxbenzylamine, methyl-2-hydroyxbenzylamine, ethyl-2-hydroyxbenzylamine.

4. The method of claim 1, wherein the compound is: 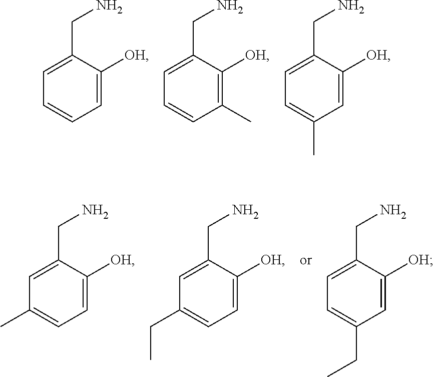
or a pharmaceutically acceptable salt thereof.

5. The method of claim 1, wherein the compound is: 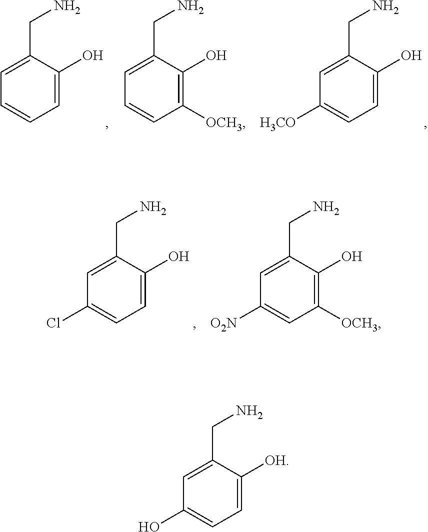
or a pharmaceutically acceptable salt thereof.

6. The method of claim 1, wherein the compound is: 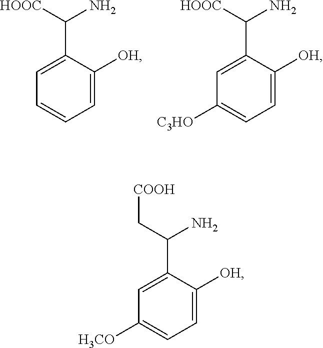
or a pharmaceutically acceptable salt thereof.

7. The method of claim 1, wherein the lupus is systemic lupus erythematosus.

8. The method of claim 1, wherein the treating step inhibits the progression of lupus.

9. The method of claim 1, wherein the treating step attenuates the severity of lupus.

10. The method of claim 1, wherein the treating step mitigates the damaging effects of lupus in the subject.

11. The method of claim 10, wherein the damaging effects include abnormal sodium excretion, augmented albuminuria, and augmented glomerular injury.

12. The method of claim 1, wherein the compound or pharmaceutically acceptable salt thereof is administered in a composition that comprises said compound or a pharmaceutically acceptable salt thereof and a pharmaceutically acceptable carrier.

13. The method of claim 1, wherein the compound or pharmaceutically acceptable salt thereof is administered is co-administered with another active agent that had a known side effect of treating lupus.
